(19) United States
(12) Patent Application Publication
(10) Pub. No.: US 2017/0183283 A1
(43) Pub. Date: Jun. 29, 2017

(54) METHOD FOR PREPARING P-HYDROXYMANDELIC COMPOUNDS IN STIRRED REACTORS

(71) Applicant: Rhodia Operations, Paris (FR)

(72) Inventors: Jean-Paul VIDAL, Vourles (FR); Pascal PITIOT, Lyon (FR)

(21) Appl. No.: 15/313,209

(22) PCT Filed: May 20, 2015
(86) PCT No.: PCT/EP2015/061135

§ 371 Date: Nov. 22, 2016

(30) Foreign Application Priority Data
May 26, 2014 (FR) 1401206

(51) Int. Cl. C07C 51/367; C07C 59/64

(52) U.S. Cl. CPC C07C 51/367; C07C 59/64

(57) ABSTRACT

The process allows the preparation of a p-hydroxymandelic compound, comprising at least one step of condensation of at least one aromatic compound bearing at least one hydroxyl group and whose para position is free, with glyoxylic acid, the condensation reaction being performed in at least one reactor equipped with at least one mixing means, the specific mixing power being between 0.1 kW/m³ and 15 kW/m³. In addition, the invention also relates to a process for preparing a 4-hydroxyaromatic aldehyde by oxidation of this p-hydroxymandelic compound.

### TECHNICAL FIELD OF THE INVENTION

The present invention relates to the preparation of p-hydroxymandelic compounds, i.e. aromatic compounds bearing at least one —CHOH—COOH group para to a hydroxyl group. More specifically, this invention relates to a process for preparing a p-hydroxymandelic compound by condensation of an aromatic compound bearing at least one hydroxyl group and whose para position is free, with glyoxylic acid.

Preferably, the p-hydroxymandelic compound is 4-hydroxy-3-methoxymandelic acid or 4-hydroxy-3-ethoxymandelic acid.

### PRIOR ART

Vanillin may be obtained from natural sources such as lignin or ferulic acid, but a substantial proportion of vanillin is produced chemically.

Numerous and various preparation methods are described in the literature (Kirk-Othmer—Encyclopedia of Chemical Technology 24, pp. 812-825, 4^{th} edition (1997)).

A conventional route of access to vanillin involves a condensation reaction of glyoxylic acid on guaiacol in basic medium to obtain 4-hydroxy-3-methoxymandelic acid. This product is then oxidized to give vanillin.

One of the technical difficulties of this reaction lies in the fact that the condensation yield is limited by its lack of selectivity. Besides p-hydroxymandelic acid, this reaction also leads to o-hydroxymandelic acid, a product derived from a parasite reaction, and also to dimandelic acids, resulting from a subsequent reaction, namely a second condensation of glyoxylic acid with a mandelic acid. In addition, glyoxylic acid may be converted via the Cannizzaro reaction into oxalic acid and glycolic acid.

For the purpose in particular of obtaining improved selectivity, international patent application WO 2009/077 383 proposes the use of a piston-flow reactor, with or without packing. This solution may, however, have certain drawbacks. Firstly, the progress of a reaction in a piston-flow reactor is generally inflexible: it is difficult to vary the reactor feed rates to a large extent, which means that the production cannot always be readily adapted to the needs. Secondly, reactors equipped with packing may become fouled.

This is why it may be preferred in certain cases to perform the condensation step in reactors equipped with stirring. However, it is desired to maintain good reaction selectivity. Preferably, it is desired to achieve at least one of these objectives:
a high degree of conversion of glyoxylic acid,
a high degree of conversion of the reactive hydroxylated aromatic compound,
a high selectivity toward the p-hydroxymandelic compound formed, relative to the hydroxylated aromatic compound converted and/or relative to the glyoxylic acid converted,
a low selectivity toward the ortho-hydroxymandelic side compound formed, relative to the hydroxylated aromatic compound converted and/or relative to the glyoxylic acid converted,
a low selectivity toward the dihydroxymandelic parasite compound formed, relative to the hydroxylated aromatic compound converted and/or relative to the glyoxylic acid converted.

Furthermore, it is not desired for this process to have an excessively high operating cost.

Patent applications WO 99/65853 and FR2495137 also disclose processes for preparing p-hydroxymandelic compounds.

It is in this context that the inventors sought a process for preparing p-hydroxymandelic compounds that can overcome one or more of the drawbacks of the piston-flow reactor mentioned above.

### BRIEF DESCRIPTION OF THE INVENTION

One subject of the invention is a process for preparing an aromatic compound bearing at least one —CHOH—COOH group para to a hydroxyl group, comprising at least one step of condensation of at least one aromatic compound bearing at least one hydroxyl group and whose para position is free, with glyoxylic acid, the condensation reaction being performed in at least one reactor equipped with at least one mixing means, the specific mixing power being between 0.1 kW/m³ and 15 kW/m³.

In addition, a subject of the invention is also a process for preparing an aromatic compound bearing at least one aldehyde group —CHO para to a hydroxyl group, this process comprising steps consisting in:
preparing an aromatic compound bearing at least one —CHOH—COOH group para to a hydroxyl group, according to the process described above, and then
oxidizing this compound.

### BRIEF DESCRIPTION OF THE FIGURES

FIG. 1 schematically represents one embodiment of the invention in which the condensation reaction is performed in a stirred reactor.

FIG. 2 schematically represents another embodiment of the invention in which the condensation reaction is performed in a reactor equipped with an external recirculation loop.

FIG. 3 schematically represents another embodiment of the invention in which the condensation reaction is performed in a cascade of stirred reactors.

FIG. 4 schematically represents another embodiment of the invention in which the condensation reaction is performed in parallel in several stirred reactors.

### DESCRIPTION OF THE INVENTION

In the description that follows, the expression “between . . . and . . . ” should be understood as including the mentioned limits.

The subject of the present invention is a process for preparing an aromatic compound bearing at least one —CHOH—COOH group para to a hydroxyl group. To do this, a condensation reaction is performed between:

on the one hand, at least one aromatic compound bearing at least one hydroxyl group and whose para position is free,

and, on the other hand, glyoxylic acid.

In the description that follows, the term “aromatic compound” especially denotes a cyclic compound bearing delocalized double bonds as defined in the literature, especially by M. Smith and J. March, Advanced Organic Chemistry, 5^{th} edition, John Wiley & Sons, 1992, pp. 46 et seq.

The reactive aromatic compound may be phenol, but also a substituted phenol having at least one position para to the hydroxyl group that is unsubstituted. The aromatic nucleus of the reactive aromatic compound bears at least one hydroxyl group, but it may also bear one or more other substituents. Generally, the term “several substituents” defines less than four substituents per aromatic nucleus. Any substituent may be present, provided that it does not interfere in the reaction of the invention.

Thus, in the process of the invention, the aromatic compound bearing at least one hydroxyl group and whose para position is free may be a compound of formula (I):

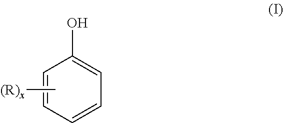

in which:

at least the position para to the hydroxyl group is free,

R represents a hydrogen atom or one or more identical or different substituents,

x is a number less than or equal to 4,

when x is greater than 1, two groups R placed on two vicinal carbon atoms may form, together with the carbon atoms that bear them, a saturated, unsaturated or aromatic ring containing from 5 to 7 atoms and optionally comprising one or more heteroatoms.

In formula (I), the identical or different groups R may represent a hydrogen atom, an alkyl, alkenyl, alkoxy, hydroxyalkyl, alkoxyalkyl, cycloalkyl, aryl or arylalkyl group, a hydroxyl group, a nitro group, a halogen atom, a halo or perhaloalkyl group, a formyl group, an acyl group containing from 2 to 6 carbon atoms, a carboxylic group, or an amino or amido group optionally substituted with one or two alkyl or phenyl groups. It should be noted that the carboxylic group may be salified, preferably with an alkali metal (sodium or potassium), or esterified, for example with an alkyl or phenyl group.

In formula (I), when x is greater than 1, two groups R placed on two vicinal carbon atoms may be linked together via an alkylene, alkenylene or alkenylidene group containing from 3 to 5 carbon atoms to form a saturated, unsaturated or aromatic ring containing from 5 to 7 atoms: one or more (preferably 2 or 3) carbon atoms possibly being replaced with a heteroatom, preferably oxygen.

Within the context of the invention, the term “alkyl” is understood to mean a linear or branched hydrocarbon-based chain having from 1 to 15 carbon atoms and preferably 1 or 2 to 10 carbon atoms. Preferred examples of alkyl groups are methyl, ethyl, n-propyl, isopropyl, n-butyl, isobutyl, sec-butyl and tert-butyl groups.

The term “alkoxy” means a group alkyl-O— in which the term “alkyl” has the meaning given above. Preferred examples of alkoxy groups are methoxy and ethoxy groups.

The term “alkenyl” means a linear or branched hydrocarbon-based group containing from 2 to 15 carbon atoms, comprising one or more double bonds, preferably 1 to 2 double bonds.

The term “cycloalkyl” means a cyclic hydrocarbon-based group comprising from 3 to 8 carbon atoms, preferably a cyclopentyl or cyclohexyl group.

The term “aryl” means a monocyclic or polycyclic, preferably monocyclic or bicyclic, aromatic group comprising from 6 to 12 carbon atoms, preferably phenyl or naphthyl.

The term “arylalkyl” means a linear or branched hydrocarbon-based group bearing a monocyclic aromatic ring and comprising from 7 to 12 carbon atoms, preferably benzyl.

The terms “halo” and “perhaloalkyl” mean one of the following groups:

—CX₃, —[CX₂]ₚ—CX₃ or —CₚHₐF_{b}; in said groups, X represents a halogen atom, preferably a chlorine or fluorine atom, p represents a number ranging from 1 to 10, b a number ranging from 3 to 21 and a+b=2 p+1.

When x is greater than 1, two groups R placed on two vicinal carbon atoms may be linked together via an alkylene, alkenylene or alkenylidene group to form a saturated, unsaturated or aromatic ring containing from 5 to 7 atoms, thus forming a bicycle with the ring of the aromatic compound. Examples of preferred bicyclic backbones are the following:

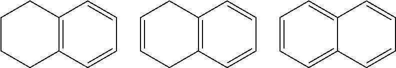

In the process of the invention, the aromatic compound bearing at least one hydroxyl group and whose para position may be advantageously chosen from the compounds of formula (I) as described above, in which R, which may be identical or different, represent:

a hydrogen atom,

a hydroxyl group,

a linear or branched alkyl group containing from 1 to 6 carbon atoms and preferably from 1 to 4 carbon atoms, such as methyl, ethyl, n-propyl, isopropyl, n-butyl, isobutyl, sec-butyl or tert-butyl,

a linear or branched alkenyl group containing from 2 to 6 carbon atoms and preferably from 2 to 4 carbon atoms, such as vinyl or allyl,

a linear or branched alkoxy group containing from 1 to 6 carbon atoms and preferably from 1 to 4 carbon atoms, such as methoxy, ethoxy, n-propoxy, isopropoxy, n-butoxy, isobutoxy, sec-butoxy or tert-butoxy,

a phenyl group,

a halogen atom, preferably a fluorine, chlorine or bromine atom.

In formula (I) the symbol “x” denotes the number of substituents on the ring, and may advantageously be a number between 0 and 4, preferably equal to 0, 1 or 2 and even more preferably equal to 1.

Preferably, in the process of the invention, the aromatic compound bearing at least one hydroxyl group and whose para position is free may be chosen from the compounds of formula (I) in which x is equal to 1 and R represents either a hydrogen atom or an alkyl group containing from 1 to 4 carbon atoms.

As illustrations of compounds corresponding to formula (I), mention may be made of:

those corresponding to formula (I) in which x is equal to 0, such as:
phenol,

those corresponding to formula (I) in which x is equal to 1, such as:
pyrocatechol
resorcinol
o-cresol
m-cresol
2-ethylphenol
3-ethylphenol
2-propylphenol
2-sec-butylphenol
2-tert-butylphenol
3-tert-butylphenol
2-methoxyphenol (also known as gaiacol)
3-methoxyphenol
2-ethoxyphenol (also known as guetol)
2-isopropoxyphenol
salicylaldehyde
methyl salicylate
2-chlorophenol
3-chlorophenol
3-nitrophenol

those corresponding to formula (I) in which x is equal to 2, such as:
2,3-dimethylphenol
2,5-dimethylphenol
3,5-dimethylphenol
2-hydroxy-5-acetamidobenzaldehy de
2-hydroxy-5-ethamidobenzaldehy de
2,3-dichlorophenol
2,5-dichlorophenol
3,5-dichlorophenol
pyrogallol

those corresponding to formula (I) in which x is equal to 3, such as:
2,3,5-trimethylphenol
3,5-di-tert-butylphenol
2,3,5-trichlorophenol

those corresponding to formula (I) bearing a naphthalene group, such as:
1-naphthol
2-naphthol
1,2-dihydroxynaphthalene
1,5-dihydroxynaphthalene
2,3-dihydroxynaphthalene
2,6-dihydroxynaphthalene
2,7-dihydroxynaphthalene
6-bromo-2-naphthol

those corresponding to formula (I) having a sequence of benzene nuclei:
2-phenoxyphenol
3-phenoxyphenol

Very preferably, in the process of the invention, the aromatic compound bearing at least one hydroxyl group and whose para position is free may be chosen from the group consisting of phenol, o-cresol, m-cresol, 3-ethylphenol, 2-tert-butylphenol, guaiacol and guetol, and even more preferably is guaiacol or guetol or a mixture thereof.

According to a preferred embodiment of the process according to the invention, it is possible for only one reactive aromatic compound to be used in the condensation step. However, it is not excluded for several reactive aromatic compounds to be used simultaneously. According to another embodiment, a mixture of several reactive aromatic compounds may be used, preferably a mixture of two reactive aromatic compounds. Very preferably, it may be a mixture of guaiacol and guetol.

In the process according to the invention, the aromatic compound bearing at least one hydroxyl group and whose para position is free undergoes a condensation reaction with glyoxylic acid. The mole ratio between the hydroxylated aromatic compound and glyoxylic acid may range between 1.0 and 4.0 and preferably between 1.2 and 2.2.

This condensation step may be performed in aqueous medium, in the presence of at least one alkaline agent.

In the case of a use in aqueous medium, the concentration of the reactive hydroxylated aromatic compound may preferably be between 0.5 and 1.5 mol/liter and more particularly about 1 mol/liter.

Glyoxylic acid may be used in aqueous solution with a concentration ranging, for example, between 15% and 70% by weight. Use is preferably made of commercial solutions whose concentration is about 50% by weight.

The alkaline agent leads to the salification firstly of the alcohol function of the hydroxylated aromatic compound, and secondly of the carboxylic function of glyoxylic acid and then of the final p-mandelic product. The alkaline agent may be an alkali metal hydroxide, especially sodium hydroxide or potassium hydroxide. For economic reasons, sodium hydroxide may be preferred. The alkali metal hydroxide solution used may have a concentration of between 10% and 50% by weight. The amount of alkali metal hydroxide introduced into the reaction medium takes into account the amount required to salify the hydroxyl function of the hydroxylated aromatic compound and the carboxylic function of glyoxylic acid. Generally, the amount of alkali metal hydroxide ranges between 80% and 120% of the stoichiometric amount.

One possible variant consists in performing the reaction in the presence of a catalyst of dicarboxylic acid type, preferably oxalic acid, as described in international patent application WO 99/65853. The amount of catalyst used, expressed by the ratio between the number of moles of catalyst and the number of moles of glyoxylic acid, may be advantageously chosen between 0.5% and 2.5% and preferably between 1% and 2%.

According to one embodiment of the present invention, the reactive hydroxylated aromatic compound and the alkaline agent are mixed together before the reactive hydroxylated aromatic compound is placed in contact with the glyoxylic acid. Thus, the process according to the invention may comprise in a first stage the placing in contact of the reactive hydroxylated aromatic compound with an alkali metal hydroxide in aqueous solution, followed by the placing in contact of the resulting solution with glyoxylic acid. This embodiment advantageously makes it possible to control the reaction temperature better, since the glyoxylic acid salification reaction is exothermic.

According to another embodiment, the process according to the invention comprises in a first stage the placing in contact of glyoxylic acid with an alkali metal hydroxide in aqueous solution, followed by the placing in contact of the resulting solution with the reactive hydroxylated aromatic compound.

According to yet another embodiment, the process according to the invention comprises, firstly, the placing in contact of the reactive hydroxylated aromatic compound with the alkaline agent in aqueous solution, and, secondly, the placing in contact of glyoxylic acid with the alkaline agent in aqueous solution, followed by the placing in contact of the two resulting solutions.

These optional steps of placing glyoxylic acid in contact with an alkali metal hydroxide in aqueous solution and/or of placing the reactive hydroxylated aromatic compound in contact with the alkaline agent may be performed at a temperature of between 10° C. and 40° C., for example at 15° C. or at 35° C.

The reaction mixture obtained may have a viscosity at 20° C. of between 0.5 mPa·s and 50 mPa·s and more preferentially between 1.5 mPa·s and 3 mPa·s. According to the invention, this mixture is introduced into at least one reactor, in which the condensation reaction takes place.

According to a first embodiment, the condensation reaction is at least partly performed in a reactor in batch regime, or in semi-continuous regime, also known as fed-batch regime. In a batch regime, there is neither input nor output of material during the reaction. In a semi-continuous regime, certain constituents are added or removed during the reaction. For example, the introduction of one or more reagents may be performed during the reaction, but there is no output of material, or vice versa. In order to be able to achieve the desired flow rates, it is possible to run several reactors in parallel. The reactors may be identical or different. From an industrial viewpoint, the production in batch or semi-batch regime may be not optimal. To overcome this difficulty, several reactors may be run in parallel and their implementations may be offset over time so as to have a more continuous production of product over time.

According to a second embodiment, the condensation reaction is at least partly performed in a reactor in continuous regime. The reaction is very preferably performed in a cascade of several reactors, preferably of at least two reactors and more preferably of at least three reactors. The number of reactors in cascade may be between 3 and 20, more preferably between 4 and 10 and even more preferably between 5 and 8. When a cascade of reactors is used, the reactors may be identical or different. It may be a cascade of stirred reactors, but not exclusively. In addition, the final reactor of the cascade of reactors may be a finishing reactor, which has a different volume from the other reactors, for example a larger volume. This finishing reactor advantageously makes it possible to adjust, according to the needs, the process output characteristics, for example the process yield and selectivity.

The operating conditions of the reaction may be set as a function of the reagents and of the type of reactor or of reactor sequence used.

The reaction temperature may be between 10° C. and 90° C. According to one embodiment, the reaction temperature may be between 10° C. and 20° C. According to another embodiment, the temperature may be between 30° C. and 40° C. Furthermore, the temperature may vary during the reaction. For example, the reaction may be performed at a temperature of between 10° C. and 20° C. for a certain time, and the temperature may then be raised to between 30° C. and 50° C. for a finishing phase. A temperature control may be carried out typically by the means of a double jacket and/or internal coils and/or equivalent means located on an external recirculation loop. The reaction may as well be carried out under adiabatic conditions. It is as well possible that the temperature is not controlled.

The reaction may be performed at atmospheric pressure, but under a controlled atmosphere of inert gases, preferably of nitrogen or, optionally, of rare gases, in particular argon. Nitrogen is preferentially chosen.

The total residence time of the reagents in a continuous regime and the operating or cycle time in a batch regime may vary widely, for example from a few minutes to several hours, or even several days, especially depending on the operating conditions, in particular depending on the reaction temperature. When the temperature is between 10° C. and 20° C., the total residence time of the reagents may be between 10 hours and 100 hours. When the temperature is between 30° C. and 50° C., the total residence time of the reagents may be between 30 minutes and 30 hours.

In the process according to the invention, the condensation reaction is performed in at least one reactor equipped with at least one mixing means. This mixing means may be chosen from the various means known to those skilled in the art.

The inventors have discovered that the mixing of the fluid inside the reactor is an important parameter for the production process according to the invention. To obtain a good yield and good selectivity during the condensation reaction of at least one aromatic compound bearing at least one hydroxyl group and whose para position is free, with glyoxylic acid, the inventors have determined that the reaction should be performed in at least one reactor equipped with at least one mixing means, to which is applied a specific mixing power of between 0.1 kW/m³ and 15 kW/m³. Preferably, the specific mixing power is between 0.1 kW/m³ and 12 kW/m³, more preferably between 0.1 kW/m³ and 10 kW/m³ and even more preferably between 0.1 kW/m³ and 5 kW/m³.

It has been found that when this power is reduced to a value below a threshold value, the condensation reaction yield drops significantly. The inventors have also discovered the existence of an upper platform value: increasing the specific mixing power beyond this threshold then brings about an overconsumption of process energy, without any benefit on the reaction yield.

In general, the specific mixing power, expressed in kilowatts per cubic meter of fluid to be mixed, may be defined as the power dissipated into the fluid, relative to the volume of fluid mixed. The power dissipated into the fluid may be estimated from the power consumed by the feed systems of the mixing means, typically motors and/or pumps. For example, dissipated power typically represents 80% of the electrical power consumed by the motor or pump at the industrial scale. Loss between consumed electrical power and power dissipated into the fluid (by mechanic friction, by temperature rise . . . ) may be measured experimentally by carrying out a blank test.

Several embodiments of the present invention are possible, depending on the mixing means used. In the present invention, the condensation reactor may be equipped with one or more mixing means, these means possibly being identical or different.

According to a first embodiment, the mixing means is a stirring rotor. The reaction is then performed in a stirred reactor. Various stirring rotor techniques are known to those skilled in the art and are commercially available. The stirring rotor may especially be chosen from the group consisting of radial rotors, axial rotors, combination rotors, twin-flow rotors, mechanical blending rotors and rotors suitable for viscous media or for media containing solids.

Among the radial rotors, examples that may be mentioned include flat-paddle turbomixers, known as Rushton mixers, incurved-paddle turbomixers, curved-paddle turbomixers and the phase-jet rotors from the company Ekato.

Among the axial rotors, examples that may be mentioned include marine impellers, thin large-paddle impellers, the rotors A310 and A320 from the company Lightnin, the rotors TTP and TT from the company Mixel, the rotors Isojet and Viscoprop-F® from the company Ekato, the rotors XE-3, HE-3 and SC-3 from the company Chemineer, and the rotors LA, LB and LC from the company Lumpp.

Among the combination rotors, examples that may be mentioned include turbomixers with 2, 4 or 6 inclined paddles.

Among the twin-flow rotors, mention may be made of the Intermig® rotors from the company Ekato.

Among the mechanical blending rotors, examples that may be mentioned include anchors, frames, impellers and the Maxblend rotor from the company Sumitomo.

Among the rotors suitable for viscous media or media containing solids, examples that may be mentioned include the helical double-band rotor and the Paravisc and Koaxial rotors from the company Ekato.

The design and size of the stirring rotor may be chosen by a person skilled in the art as a function of the desired mixing performance. The size of the rotor may be chosen especially such that the D (rotor diameter)/T (reactor diameter) ratio is between 0.3 and 0.7 in the case of radial, axial or combination rotors. In the case of mechanical blending rotors, the ratio D/T may more preferentially be between 0.9 and 1.

The person skilled in the art may associate internals to the stirring rotor, typically baffles, or any other means having the same function (coil or temperature regulation pin . . . ).

According to this embodiment, the specific mixing power is equal to the specific power delivered by the stirring rotor. This may be calculated from the knowledge of the technical data supplied by the manufacturer of the stirring rotor, the spin speed of the rotor, the rotor diameter, the mass per unit volume of the fluid to the mixed and the volume of the liquid to be mixed. The following formulae may especially be applied:

$$∈ = \frac{P}{V} = \frac{{N}_{p} \times ρ \times {N}^{3} \times {D}^{5}}{V}$$

in which:
ε is the specific power, in watts/m³,
P is the power in watts,
V is the volume of the liquid to be mixed, in m³,
Np is the power number, i.e. a dimensionless and tabulated value given by the constructor as a function of the rotor and of the hydrodynamic conditions,
ρ is the mass per unit volume of the fluid to be mixed, in kg/m³,
N is the stirring speed, in Hz, and
D is the rotor diameter, in m.

Power number Np depends in particular on Reynolds Number (Re) which characterizes the hydrodynamic state of the mixed liquid:

$$Re = \frac{ρ \times N \times {D}^{2}}{μ}$$

with ρ, N, and D as defined above and μ the viscosity of the liquid, in Pa·s.

When several rotors are arranged on the same stirring shaft, their specific powers add together.

The specific stirring power per unit of mass, in watts/kg, corresponds to the specific power (in watts/m³) divided by the mass per unit volume of the fluid.

In practice, the specific stirring power delivered by a rotor may be calculated from the stirring power P in watts, i.e. the power dissipated in the mixed fluid, divided by the volume of fluid contained in the reactor. The stirring power P may be estimated from the power consumed by the motor which drives the rotor, for example the electrical power consumed by the motor. P typically represents 80% of the power consumed by the motor at the industrial scale.

According to this first embodiment, the condensation reaction is performed in at least one stirred reactor equipped with a stirring rotor, the specific stirring power being between 0.1 kW/m³ and 15 kW/m³. More preferably, the specific stirring power is less than 10 kW/m³, and even more preferably the specific stirring power is less than 5 kW/m³. For standard stirring rotors, the specific stirring power is more preferably between 0.5 kW/m³ and 10 kW/m³ and even more preferably between 0.3 kW/m³ and 5 kW/m³. For stirring rotors with low energy consumption, which are known as hydrofoils, the specific stirring power is more preferably between 0.1 kW/m³ and 5 kW/m³ and even more preferably between 0.1 kW/m³ and 2 kW/m³. The axial rotors A310 and A320 from the company Lightnin, the axial rotors TTP and TT from the company Mixel, the axial rotors Isojet and Viscoprop-F® from the company Ekato and the axial rotors XE-3, HE-3 and SC-3 from the company Chemineer are included among hydrofoils.

As an alternative to the stirring rotors, the mixing of the reaction medium may be performed by other mixing means, which may be considered as equivalent to the stirring rotors.

According to a second embodiment of the present invention, the mixing means is an external recirculation circuit. The reactor in which the condensation reaction is performed is, in this case, equipped with at least one external recirculation circuit equipped with a pump. Under the action of this pump, part of the fluid contained in the reactor is withdrawn, circulated through an external pipe and reinjected into the reactor. This movement ensures the mixing of this part of the fluid, and more generally of all the fluid contained in the reactor. It is possible to provide a heat exchanger on the external recirculation pipe, so as to keep the fluid at a desired temperature. In addition or alternatively, another mixing device, for example a rotor-stator, may be arranged on the external recirculation type. According to this embodiment, the reactor may be run in batch, semi-continuous or continuous regime.

According to a third embodiment, the reactor mixing means according to the invention is an oscillating device. The company DRM especially sells oscillating rotors under the brand name Fundamix®. Oscillation of the rotor in the reactor ensures mixing of the reaction medium. Another type of oscillating device consists of a pulsed column, for example a COBR reactor (continuous oscillatory baffled reactor). Such reactors are sold, for example, by the company Nitech. According to this embodiment, the reactor may be run in batch, semi-continuous or continuous regime. When it is a reactor of pulsed column type, the process may be performed in batch regime by means of a recycling loop between the reactor inlet and outlet.

According to a fourth embodiment, the reactor equipped with a mixing means according to the invention is a planetary mixer. A planetary mixer consists of a reactor equipped with both a disperser and an overall recirculation rotor. According to this embodiment, the reactor may be run in batch, semi-continuous or continuous regime.

In the above three cases, the power deployed by the mixing means is not dissipated into all of the fluid, but into a certain volume of this fluid. For example, when the mixing means is an external recirculation circuit, only the fluid contained in the volume of the pump of the receives the power delivered by the pump. However, for the purposes of the present invention, the specific mixing power of these embodiments is defined as the power produced by the mixing means dissipated into the fluid divided by the total volume of fluid. Thus, when the mixing means is an external recirculation circuit, the specific mixing power within the meaning of the invention is the power produced by the recirculation pump dissipated into the fluid divided by the total volume of fluid in the reactor. When the mixing means is an oscillating rotor, the specific mixing power within the meaning of the invention is the power dissipated into the fluid produced by the motor of the rotor divided by the total volume of fluid in the reactor. When the mixing means is a COBR, the specific mixing power within the meaning of the invention is the total power dissipated into the fluid produced by the pump driving the fluid in motion in the reactor and by the device generating the pulses divided by the total volume of fluid in the reactor. Finally, when the reaction is performed in a planetary mixer, the specific mixing power within the meaning of the invention is the power dissipated into the fluid produced by the motor of the disperser and of the recirculation rotor divided by the total volume of fluid in the reactor.

Finally, according to a fifth embodiment, the mixing means consists of structuring of the reactor. The condensation reaction is then performed in a structured reactor. The structuring may be chosen from 2D or 3D static mixers, metallic or ceramic foams, bulk packing and geometrical structurings. Companies such as Sulzer, Kenics-Chemineer and Verder propose numerous types of structuring. Moreover, the reaction may be performed in a thin straight tube. According to this embodiment the specific mixing power within the meaning of the invention is the power dissipated into the fluid produced by the pump driving the fluid in motion in the reactor divided by the total volume of fluid in the reactor. This specific power may be deduced here by calculating the pressure losses suffered by the fluid in the structured reactor. Preferably, in this embodiment, the reactor is run in batch regime, which is made possible by means of a recycling loop between the reactor inlet and outlet.

At the end of the reaction, an aromatic compound bearing at least one —CHOH—COOH group para to a hydroxyl group is obtained. This compound may be chosen from the compounds represented by formula (II) below:

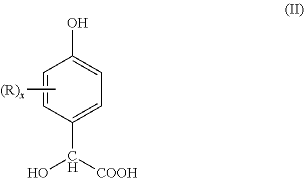

in which R and x have the meanings given in formula (I).

Preferably, the aromatic compounds bearing at least one —CHOH—COOH group para to a hydroxyl group is chosen from 4-hydroxy-3-methoxymandelic acid and 4-hydroxy-3-ethoxymandelic acid, and a mixture thereof.

According to a specific embodiment, the process according to the invention is a process for preparing 4-hydroxy-3-methoxy-mandelic acid, 4-hydroxy-3-ethoxy-mandelic acid, or a mixture thereof, comprising at least a step of condensation of guaiacol, of guethol, or of the mixture thereof, with glyoxylic acid.

When the reaction is performed in aqueous medium in the presence of an alkaline agent, the p-hydroxymandelic compound obtained may be in salified form. A neutralization step may then be performed in order to obtain the p-hydroxymandelic compound bearing an acid function as described above.

These products are particularly advantageous since they are intermediate products for obtaining, inter alia, by reduction, hydroxyarylacetic acids, or, by oxidation, hydroxyarylglyoxylic acids (i.e. hydroxyaryl α-oxoacetic acids) or hydroxyaromatic aldehydes.

A preferred application of the invention is the preparation of hydroxyaromatic aldehydes, via oxidation of the compounds of formula (II) obtained according to the invention.

After the condensation reaction, the p-hydroxymandelic compound obtained may be separated from the reaction mixture via standard separation techniques, especially by crystallization or by extraction using a suitable organic solvent. A neutralization step may be performed. Alternatively, the reaction mixture obtained after the condensation reaction may be used in its existing form. However, it is preferable to recover the unreacted hydroxylated aromatic compound.

To this end, use may be made of the treatments described in the prior art, especially the treatments described in patent FR 2 379 501. It consists in adding a mineral acid, for example hydrochloric acid or sulfuric acid, to adjust the pH to between 5 and 7, and then in extracting the unreacted hydroxylated aromatic compound in an organic solvent, especially in ether or toluene. After extraction, the aqueous and organic phases may be separated.

A subject of the present invention is also a process for preparing an aromatic compound bearing at least one aldehyde group —CHO para to a hydroxyl group, this process comprising steps consisting in:
preparing an aromatic compound bearing at least one —CHOH—COOH group para to a hydroxyl group, according to the process described previously, and then
oxidizing this compound.

The oxidation may be performed according to the techniques described in the literature. Thus, reference may be made to P. Hebert (Bull. Soc. Chim. France, 27, pp. 45-55, 1920) and to Nagai Shigeki et al., (JP-A 76/128 934). The oxidation is generally performed under an oxidizing atmosphere, such as oxygen or air, in basic medium and in the presence of a suitable catalyst. The word “oxidation” here refers to a decarboxylative oxidation, since it comprises the leaving a carboxylate group, forming carbon dioxide.

Thus, the invention affords easy access to 4-hydroxybenzaldehyde and to vanillin and analogs thereof, for example 3-ethylvanillin or 3-isopropylvanillin, by oxidation, respectively, of p-hydroxymandelic acid and of 4-hydroxy-3-methoxymandelic, 3-ethoxy-4-hydroxymandelic and 4-hydroxy-3-isopropoxymandelic acids.

According to a specific embodiment, the process according to the invention is a process for preparing vanillin, ethylvanillin or mixture thereof, said process comprising the steps of:
Preparing 4-hydroxy-3-methoxy-mandelic acid, 4-hydroxy-3-ethoxy-mandelic acid or mixture thereof, according to the process disclosed above, then Oxidizing said compound.

### EXAMPLES

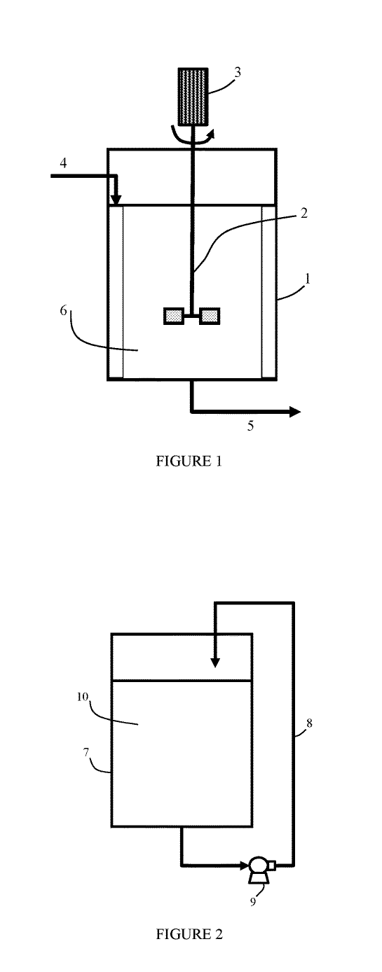

FIG. 1 schematically represents one embodiment of the invention in which the condensation reaction is performed in a stirred reactor. Reactor 1 is equipped with a stirring means which consists of a stirring rotor 2. This rotor is driven by a motor 3. The stirring rotor 2 allows the reaction medium 6 to be mixed. In FIG. 1, the reactor 1 is run in continuous regime: the reagents are introduced via the inlet 4 and are removed via the outlet 5. However, the invention is not limited to this embodiment, and this stirred reactor may also be used in batch or semi-continuous regime.

FIG. 2 schematically represents another embodiment of the invention in which the condensation reaction is performed in a reactor 7 equipped with an external recirculation circuit 8. The fluid is placed in motion in the external recirculation circuit 8 by means of the pump 9. The external recirculation circuit 8 ensures mixing of the reaction medium 10.

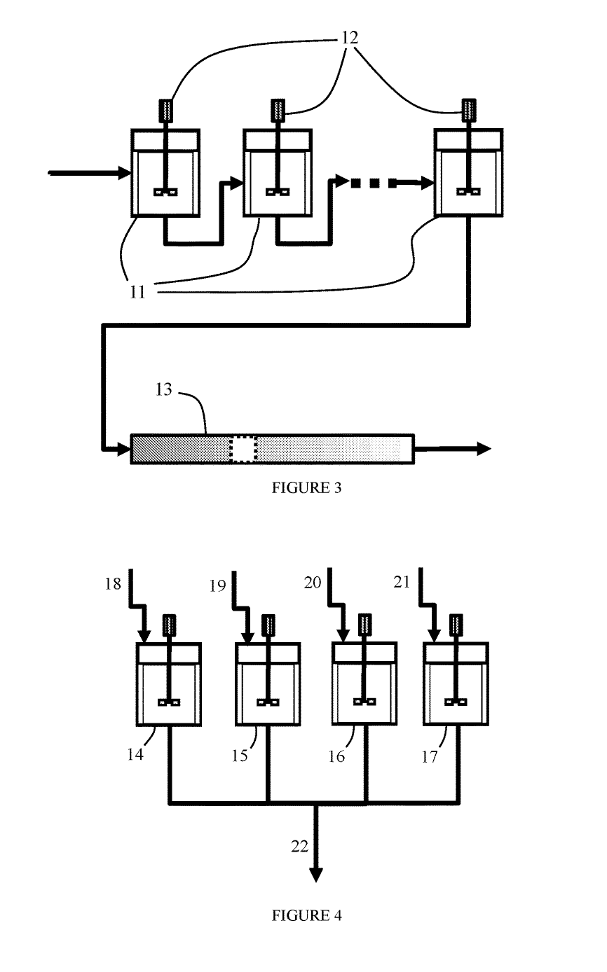

FIG. 3 schematically represents another embodiment of the invention in which the condensation reaction is performed in a cascade of stirred reactors 11. Each reactor 11 is equipped with a mixing means, in this case a stirring rotor 12. In this FIG. 3, three stirred reactors 11 are shown. However, this number is not limited to three. At the outlet of the last stirred reactor 11, the reaction medium is introduced into a finishing reactor 13. In FIG. 3, the finishing reactor 13 is a tubular reactor, with piston-type flow. However, the invention is not limited to this embodiment, and the finishing reactor may be, for example, a stirred reactor.

FIG. 4 schematically represents another embodiment of the invention in which the condensation reaction is performed in parallel in several stirred reactors. The stirred reactors 14, 15, 16 and 17 are arranged in parallel and are run in batch or semi-continuous regime, each being fed, respectively, with reagents via the pipes 18, 19, 20 and 21. By means of the parallel running of the four reactors, the stream of products obtained at 22 may be evened out.

### Examples 1 to 3

A device according to FIG. 1 was provided with a 10 L glass reactor mounted with four 316 L stainless steel-baffles. The mixing rotor was a Lightnin® A310 rotor having a diameter of 14cm. The reactor was further provided with a double jacket for controlling the temperature, a pH electrode, a temperature probe linked to the control. We introduces into this reactor:

6000 g distilled water,

1300 g soda solution (30 wt. %),

800 g guaiacol,

555.0 g of an aqueous solution of the glyoxilic acid (50 wt. %).

Under inert atmosphere, the temperature was set to 38° C. and the reaction was carried out during 100 min under stirring. Stirring speed N and stirring specific power ε are as mentioned in the table below.

At the end of the reaction, the products were titrated by HPLC. The conversion rate of guaiacol Cony. (GA) (number of moles of transformed guaiacol vs. number of moles of introduced guaiacol) and the yield of 4-hydroxy,3-methoxy-mandelic acid RR(APM)/GA (number of moles of formed 4-hydroxy,3-methoxy-mandelic acid vs. number of moles of introduced guaiacol) were determined.

| | Example 1 | | Example 2 | | Example 3 | |
|---|---|---|---|---|---|---|
| Stirring speed N | 180 | rpm | 330 | rpm | 625 | rpm |
| Stirring specific power | 0.05 | kW/m³ | 0.3 | kW/m³ | 2.0 | kW/m³ |
| ε* | | | | | | |

| | | | |
|---|---|---|---|
| Conv. (GA) | 28.2% | 53.1% | 53.3% |
| RR(APM)/GA | 21.8% | 45.9% | 46.1% |

* The stirring specific power ε was calculated with the formula

$$∈ = \frac{{N}_{p} \times ρ \times {N}^{3} \times {D}^{5}}{V}$$

(power number Np=0.30, density of the mixed liquid ρ=1100kg/m³, rotor diameter D=0.14 m, volume of the mixed liquid V=10×10⁻³ m³).

### Example 4

The stirring rotor of Example 1 was replaced by a Rushton mixer having a diameter of 11,5cm. In these conditions, the power number Np is 5,5. The mixing speed was set to 180 rpm, so that the mixing specific power ε is 0.33 kW/m³.

The reaction of Example 1 was reproduced.

A guaiacol conversion of 53.5% was obtained and the yield of 4-hydroxy,3-methoxy-mandelic acid was 46.0%.

### Example 5

A device according to FIG. 2 was provided with the reactor disclosed in Example 1, but the mixing rotor was replaced by an external recirculation loop. The reaction medium was pumped from the valve at the bottom of the reactor, and reinjected into the reactor via a plunging tube extending at 5cm of the (internal) bottom of the reactor, along by one of the above-mentioned baffles. To improve the local mixing, the plunging tube was leant of 45° when compared to the horizontal on 5cm. With a flow of 14 L/min, the specific power of the mixing produced by the pump is 0.1 kW/m³.

The reaction of Example 1 was reproduced.

A guaiacol conversion of 53.0% was obtained and the yield of 4-hydroxy,3-methoxy-mandelic acid was 45.7%.

1. A process for preparing an aromatic compound bearing at least one —CHOH—COOH group para to a hydroxyl group, comprising condensing, in a condensation reaction performed in at least one reactor equipped with at least one mixer having a specific mixing power of between 0.1 kW/m³ and 15 kW/m³, at least one aromatic compound bearing at least one hydroxyl group and whose position para to the hydroxyl group is free, with glyoxylic acid.

2. The process as claimed in claim 1, characterized in that the at least one aromatic compound is selected from the group consisting of phenol, o-cresol, m-cresol, 3-ethylphenol, 2-tert-butylphenol, guaiacol and guetol.

3. The process as claimed in claim 1, wherein the condensation reaction is performed in a batch regime or in semi-continuous regime.

4. The process as claimed in claim 3, wherein several reactors are run in parallel.

5. The process as claimed in claim 1, wherein the condensation reaction is performed in a reactor in continuous regime.

6. The process as claimed in claim 5, claim 1, wherein the condensation reaction is performed in a cascade of several reactors.

7. The process as claimed in claim 6, wherein the last reactor of the cascade of reactors is a finishing reactor.

8. The process as claimed in claim 1, wherein the specific mixing power of the mixer is between 0.1 kW/m³ and 12 kW/m³.

9. The process as claimed in claim 1, wherein the mixer comprises a stirring rotor.

10. A process for preparing an aromatic compound bearing at least one aldehyde group —CHO para to a hydroxyl group, comprising:
preparing an aromatic compound bearing at least one —CHOH—COOH group para to a hydroxyl group, according to the process of claim 1, and
oxidizing the aromatic compound bearing at least one —CHOH—COOH group para to a hydroxyl group.

11. The process of claim 2, wherein the at least one aromatic compound is selected from the group consisting of guaiacol, guetol, and mixtures thereof.

12. The process of claim 6, wherein the condensation reaction is performed in a cascade of at least two reactors.

13. The process of claim 6, wherein the condensation reaction is performed in a cascade of at least three reactors.

14. The process as claimed in claim 8, wherein the specific mixing power of the mixer is between 0.1 kW/m³ and 10 kW/m³.

15. The process as claimed in claim 8, wherein the specific mixing power of the mixer is between 0.1 kW/m³ and 5 kW/m³.

16. The process as claimed in claim 9, wherein the stirring rotor is selected from the group consisting of radial rotors, axial rotors, combination rotors, mechanical blending rotors, and rotors suitable for viscous media or media containing solids.
